(19) United States
(12) Patent Application Publication
(10) Pub. No.: US 2025/0144126 A1
(43) Pub. Date: May 8, 2025

(54) APPLICATION OF 5'-MONOPHOSPHATE NUCLEOTIDE AND MIXTURE THEREOF IN PREPARATION OF DRUG OR FOOD FOR IMPROVING MITOCHONDRIAL FUNCTION

(71) Applicant: Yusong CHEN, Dalian, Liaoning (CN)

(72) Inventors: Yong LI, Dalian, Liaoning (CN); Na ZHU, Dalian, Liaoning (CN); Meihong XU, Dalian, Liaoning (CN); Yusong CHEN, Dalian, Liaoning (CN)

(21) Appl. No.: 18/291,328

(22) PCT Filed: Jun. 10, 2022
(86) PCT No.: PCT/CN2022/098181

§ 371 Date: Apr. 22, 2024

(30) Foreign Application Priority Data
Jul. 27, 2021 (CN) 202110851454.X

(51) Int. Cl. A61K 31/7076; A23L 33/13; A61K 31/7072; A61P 39/06

(52) U.S. Cl. CPC A61K 31/7076; A23L 33/13; A61K 31/7072; A61P 39/06

(57) ABSTRACT

5′-monophosphate nucleotide and a mixture thereof are used in preparation of a drug or food for improving a mitochondrial function. 5′-Monophosphate nucleotide is a mixture of a 5′-adenosine monophosphate and 5′-disodium uridine which are produced by means of enzymatic degradation of a ribonucleic acid as a raw material and has a purity of 99% or more. The 5′ nucleotide mixture contains: 23-78% of CMP, 6-44% of AMP, 7-40% of UMP, 7-51% of GMP, and IMP being 0, or greater than 0 and not higher than 2.5%. It has been found 5′-monophosphate nucleotide and the mixture thereof can improve the mitochondrial function of H₂O₂-induced senescence fibroblasts.

### TECHNICAL FIELD

The present invention belongs to the field of medicine and health care, and relates to a nucleotide product and a new application thereof, in particular to an application of two 5′-monophosphate nucleotides and mixture thereof, produced by means of enzymatic degradation of ribonucleic acid as raw material, in drugs or foods.

### BACKGROUND

In recent ten years, a large number of studies have shown that there is a causal relationship between mitochondrial dysfunction and major phenotypes related to senescence. Disturbance of mitochondrial homeostasis is an important feature of cellular senescence. Since the theories of free radical senescence and mitochondrial senescence were proposed, mitochondria have become the focus of senescence research. According to free radical theory, ROS continuously produced during metabolism triggers DNA chain free radical reaction, causing denaturation and crosslinking of DNA, nucleic acid, protein and lipids, especially polyunsaturated fatty acids and other macromolecular substances, damaging DNA, biofilm, important structural proteins and functional proteins, and making cells unable to play normal functions, thus causing senescence. Mitochondrial senescence theory holds that mitochondrion is the organelle with the highest concentration of free radicals, and mitochondrial DNA (mtDNA) is prone to mutation when exposed to matrix.

Mitochondria are vital to life and participate in ATP production, apoptosis, β oxidation of fatty acids and other important physiological processes. Mammalian mitochondrial genome encodes 13 proteins, 22 tRNAs, and 2 rRNAs. Mammalian mitochondrial proteome includes more than 1200 kinds of proteins, almost all of which are encoded by nuclear DNA, and then introduced into mitochondria after being released to cytoplasm. Mitochondrial DNA only encodes 1.1% of mitochondrial proteome, but these proteins are the key components of oxidative phosphorylation (OXPHOS) complexes, which are essential for mitochondria to play a normal function. The morphology, abundance, and OXPHOS activity of mitochondria change with age. Studies have reported that, in senescent cells, the volume and number of mitochondria increase, mtDNA mutates, and copy number increases by 2 to 4 times; and reducing mitochondrial or mtDNA content can antagonize senescence and senescence-associated secretory phenotype (SASP). Studies suggest that continuous accumulation of mtDNA damage may be related to senescence, mutation of mtDNA may result in severe damage to cell energy conversion and tissue dysfunction, including increased mitochondrial fragility, membrane potential disorder, respiratory chain dysfunction, decreased activity of electron transport chain complexes, and increased oxidative damage. Mutation of mtDNA impairs the function of respiratory chain and further causes the accumulation of free radicals, resulting in senescence. Chinese patent (CN108697722B) disclosed that β-nicotinamide mononucleotide nutritional supplements or drugs can prevent or treat mitochondrial dysfunction-related diseases. Chinese patent (CN105073766A) disclosed that 20%-35% of nucleotides and oligonucleotides contained in extra-concentrated flavonoids can repair genes of cells and mitochondria thereof. However, there is no related patent on improving cellular mitochondrial function with 5′-monophosphate nucleotides or their mixtures with different proportions obtained by biological enzymatic hydrolysis technology.

Nucleotide is composed of pentose, base and phosphate, which is the basic unit of nucleic acid. Nucleotides can be derived from endogenous synthesis in human body and exogenous synthesis such as biological enzymatic hydrolysis technology. Exogenous nucleotides are indispensable nutritional ingredients under specific physiological conditions. In tissues and organs with vigorous metabolism or when the body is subjected to stress, immune challenge, liver injury, hunger and rapid growth, nucleotides can be absorbed and utilized by tissues, saving the body's consumption from de novo synthesis or remedial synthesis, thereby optimizing the tissue function. In addition, the decomposition process in vivo can be omitted after nucleic acid is degraded into nucleotides by enzymatic hydrolysis in vitro, which is easier to be digested and absorbed by human body. At present, there is no report on the effect of exogenous nucleotides obtained by enzymatic hydrolysis technology on improving mitochondrial function of senescent cells.

### SUMMARY OF THE INVENTION

An object of the present invention is to provide an application of two 5′-monophosphate nucleotides with low molecular weight and fast absorption and mixtures thereof in preparation of drugs or foods for improving mitochondrial function.

In order to realize the object, the present invention adopts the following technical solutions:

One aspect of the present invention provides an application of 5′-monophosphate nucleotide in preparation of a drug or food for improving mitochondrial function. The 5′-monophosphate nucleotide include two 5′-monophosphate nucleotides, which is 5′-adenosine monophosphate or 5′-disodium uridine.

Further, the 5′-adenosine monophosphate and 5′-disodium uridine are exogenous nucleotide 5′-adenosine monophosphate and 5′-disodium uridine produced by means of enzymatic degradation of a ribonucleic acid as a raw material, having a purity of more than 99%.

Another aspect of the present invention provides an application of a nucleotide mixture in preparation of a drug or food for improving mitochondrial function. The nucleotide mixture includes four or five exogenous nucleotides or sodium salts thereof, and mass percentages of the various nucleotides calculated according to forms of CMP, AMP, UPMP, GMP, IMP are respectively as follows: 23-78% of CMP, 6-44% of AMP, 7-40% of UMP, 7-51% of GMP, and IMP being 0 or greater than 0 and not greater than 2.5%.

Further, the mass percentages of the various nucleotides calculated according to forms of CMP, AMP, UPMP, GMP, IMP are respectively as follows: 25.80% of CMP, 22.80% of AMP, 20.40% of UMP, 30.20% of GMP and 0.8% of IMP.

Further, for the application of 5′-monophosphate nucleotide and a mixture thereof in preparation of a drug or food for improving mitochondrial function, the concentrations of the 5′-monophosphate nucleotide and the mixture thereof (5′-monophosphate nucleotide or nucleotide mixture) is 100-200 μmol/L.

Further, the drugs of the present invention are in the forms of powders, tablets, soft capsules, hard capsules, or oral liquids.

Further, the foods of the present invention are in the forms of powders or liquid beverages, preferably in the forms of milk powders, dairy products, or bakery products.

Further, the 5′-monophosphate nucleotide and the mixture thereof significantly improve a basal respiration, a maximum respiratory value, an ATP production capability and a respiratory reserve of the mitochondria of H₂O₂-induced senescent cells.

The present invention finds that two kinds of 5′-monophosphate nucleotides and mixtures thereof have the function of improving the mitochondrial function of H₂O₂-induced senescence fibroblasts, and finds the application thereof in preparation of drugs or foods for improving mitochondrial function. Cell experiments prove that the said two kinds of 5′-monophosphate nucleotides and mixtures thereof can significantly improve the basal respiration, maximum respiratory value, ATP production capability and respiratory reserve of mitochondria of H₂O₂-induced senescent cells.

DETAILED DESCRIPTION OF DRAWINGS

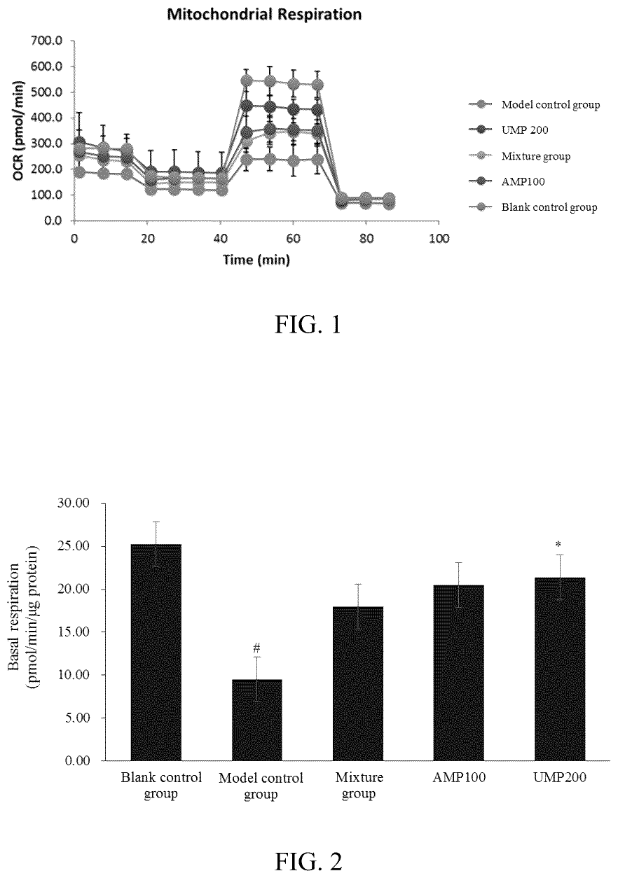

FIG. 1 shows effects of 5′-monophosphate nucleotides and mixtures thereof on mitochondrial oxygen consumption rate of senescent cells in Examples 1 and 2.

FIG. 2 shows effects of 5′-monophosphate nucleotides and mixtures thereof on mitochondrial basal respiration of senescent cells in Examples 1 and 2.

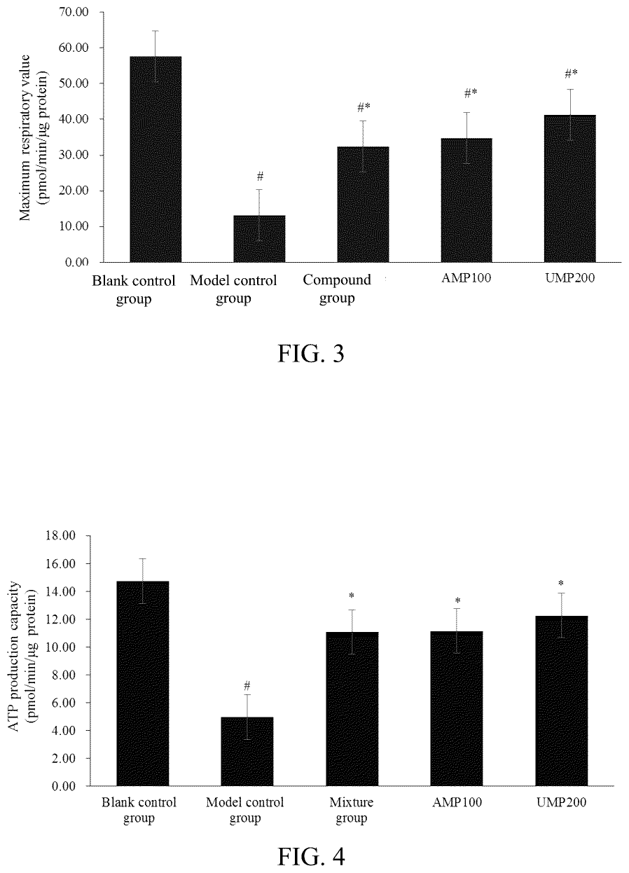

FIG. 3 shows effects of 5′-monophosphate nucleotides and mixtures thereof on mitochondrial maximum respiratory value of senescent cells in Examples 1 and 2.

FIG. 4 shows effects of 5′-monophosphate nucleotides and mixtures thereof on mitochondrial ATP production capability of senescent cells in Examples 1 and 2.

FIG. 5 shows effects of 5′-monophosphate nucleotides and mixtures thereof on

mitochondrial respiratory reserve of senescent cells in Examples 1 and 2.

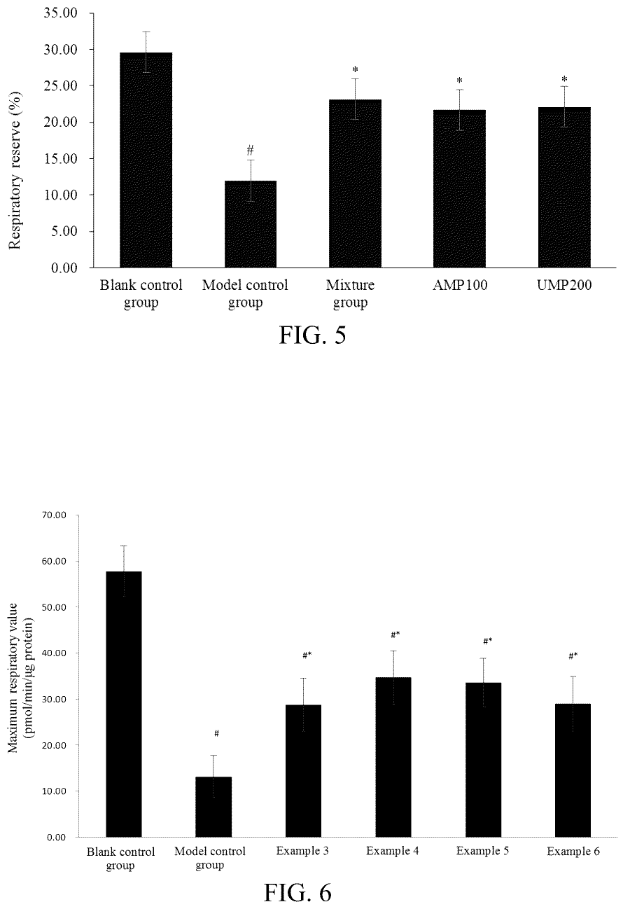

FIG. 6 shows effects of the 5′-monophosphate nucleotide mixtures on mitochondrial maximum respiratory value of senescent cells in Examples 3-6.

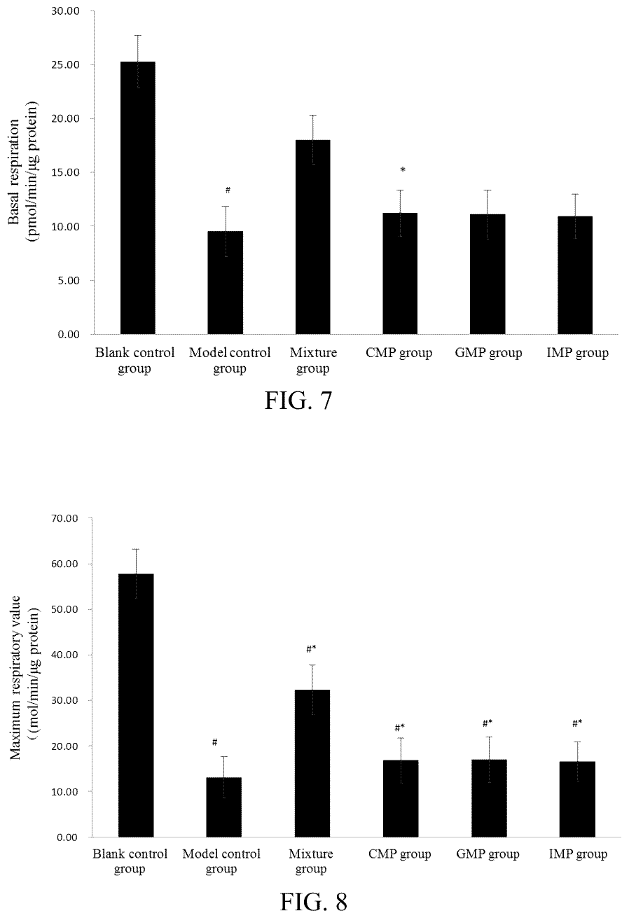

FIG. 7 shows effects of a 5′-monophosphate nucleotide mixture and single nucleotide species (CMP, GMP, IMP) on mitochondrial basal respiration of senescent cells in Example 2.

FIG. 8 shows effects of a 5′-monophosphate nucleotide mixture and single nucleotide species (CMP, GMP, IMP) on mitochondrial maximum respiratory value of senescent cells in Example 2.

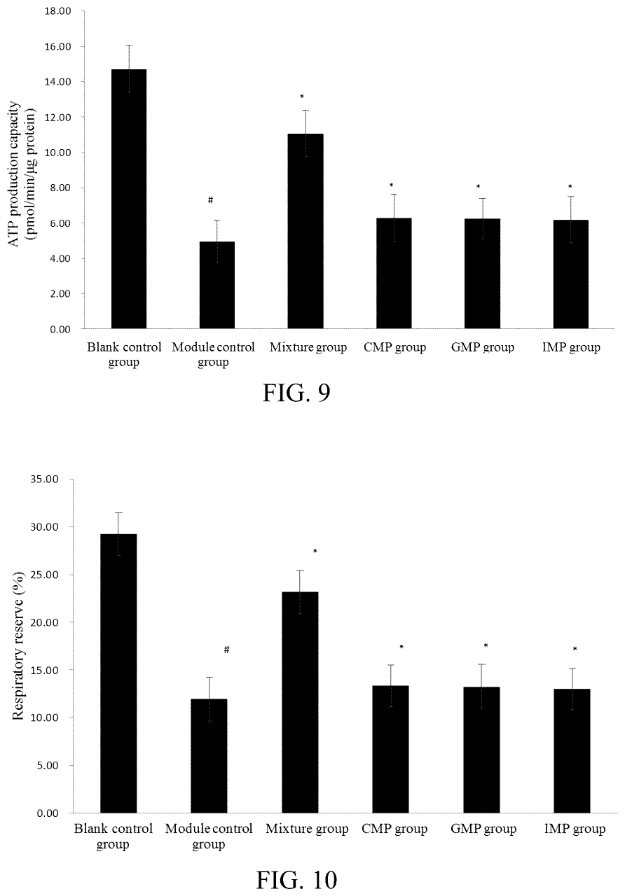

FIG. 9 shows effects of a 5′-monophosphate nucleotide mixture and single nucleotide species (CMP, GMP, IMP) on mitochondrial ATP production capability of senescent cells in Example 2.

FIG. 10 shows effects of a 5′-monophosphate nucleotide mixture and single nucleotide species (CMP, GMP, IMP) on mitochondrial respiratory reserve of senescent cells in Example 2.

### DETAILED DESCRIPTION OF PREFERRED EMBODIMENTS

The present invention will be further described below in conjunction with specific embodiments, which should be understood to be illustrative only and not intended to limit the scope of the present invention in any way.

### Example 1

1. The two 5′-monophosphate nucleotides of this example are respectively 5′-adenosine monophosphate and 5′-disodium uridine.
2. Production process: The said two 5′-monophosphate nucleotides were prepared, using ribonucleic acid (RNA) with a purity of 90% as raw material, by thermal denaturation, enzymolysis with nuclease P1, μltrafiltration, adsorption on anion resin, step-by-step elution, nanofiltration desalination, concentration with film, decolorization with carbon powder, crystallization, and drying. The obtained two 5′-monophosphate nucleotides are respectively detected, and qualified nucleotides are ready for later use.

### Example 2

1. The 5′-monophosphate nucleotide mixture of this example was a mixture of five 5′-mononucleotides or sodium salts thereof. The mixture was obtained at the following percentages: 25.80 wt % of CMP, 22.80 wt % of AMP, 20.40 wt % of UMP, 30.20 wt % of GMP and 0.8 wt % of IMP.
2. Preparation method is as follows:

The four or five 5′-mononucleotides or sodium salts thereof were detected respectively, and the qualified were ready for later use.

The qualified four or five 5′-mononucleotides or sodium salts thereof were passed through a 60-mesh sieve, and the undersize were ready for later use.

The required amount of each of the mononucleotides was calculated and weighed according to the percentages, and then all of which were fully mixed for no less than 40 minutes. The obtained sample was stored at room temperature.

### Example 3

The 5′-monophosphate nucleotide mixture of this example was a mixture of five 5′-mononucleotides or sodium salts thereof. The mixture was obtained at the following percentages: 78 wt % of CMP, 6 wt % of AMP, 7 wt % of UMP, 7 wt % of GMP and 2 wt % of IMP. The preparation method was the same as that in Example 2.

### Example 4

The 5′-monophosphate nucleotide mixture of this example was a mixture of five 5′-mononucleotides or sodium salts thereof. The mixture was obtained at the following percentages: 23 wt % of CMP, 44 wt % of AMP, 25 wt % of UMP, 7 wt % of GMP and 1.0 wt % of IMP. The preparation method was the same as that in Example 2.

### Example 5

The 5′-monophosphate nucleotide mixture of this example was a mixture of five 5′-mononucleotides or sodium salts thereof. The mixture was obtained at the following percentages: 23 wt % of CMP, 17 wt % of AMP, 40 wt % of UMP, 19 wt % of GMP and 1 wt % of IMP. The preparation method was the same as that in Example 2.

### Example 6

The 5′-monophosphate nucleotide mixture of this example was a mixture of five 5′-mononucleotides or sodium salts thereof. The mixture was obtained at the following percentages: 24 wt % of CMP, 17 wt % of AMP, 7 wt % of UMP, 51 wt % of GMP and 1 wt % of IMP. The preparation method was the same as that in Example 2.

### Example 7

### I. Materials and Methods

### 1. Samples

Mixtures of 5′-adenosine monophosphate, 5′-disodium uridine and 5′-monophosphate nucleotide obtained in Examples 1-6.

### 2. Groups and Dosages

In this example, concentrations of the 5′-adenosine monophosphate and the 5′-disodium uridine obtained in Example 1 were 100 μmol/L and 200 μmol/L respectively, and were recorded as AMP 100 group and UMP 200 group, respectively. Concentration of each the nucleotide mixture obtained in Examples 2-6 was 200 μmol/L, which was recorded as nucleotide mixture group. Model control group was a senescence cell model group intervened by hydrogen peroxide. Blank control group was a group free of mixture of 5′-adenosine monophosphate, 5′-disodium uridine and 5′-monophosphate nucleotide and without hydrogen peroxide intervention.

### 3. Experimental Cells

The cells used in the example were mouse embryonic fibroblasts NIH/3T3, which were purchased from Cell Bank of Chinese Academy of Sciences.

### 4. Establishment of Senescence Cell Model

The cells were cultured in a DMEM high-sugar medium containing 1% penicillin/streptomycin and 10% fetal bovine serum in an incubator with 5% of CO₂ at 37° C. under saturated humidity conditions. The establishment of the senescence cell model was as follows: After the cells grew to the wall of a culture vessel, different concentrations of hydrogen peroxide were used for intervention, where the concentrations of hydrogen peroxide were 50 μmol/L, 100 μmol/L, 200 μmol/L, 400 μmol/L, and 800 μmol/L. The medium containing hydrogen peroxide was removed after incubation for 4 hours, and a normal complete medium (DMEM high-sugar medium containing 1% penicillin/streptomycin and 10% fetal bovine serum) was added for incubation for 24 hours, then the cell activity of the cultured cells was detected by using a CCK-8 cell active kit, the expression of β-galactosidase was detected, and the expressions of the senescent marker p16INK4A and p21Waf1/Cipl were detected by western blot method. According to the senescent result, 200 μmol/L was selected as the optimal concentration of hydrogen peroxide for subsequent experiments.

### 5. Experimental Method

In this example, the oxygen consumption rate (OCR), basal respiration, maximum respiratory value, ATP production capability and respiratory reserve of mitochondria were determined by using the Seahorse xFe96 (Agilent) mitochondria stress detection experiment. 20000 NIH/3T3 cells were inoculated in each well of a 96-well cell culture plate (Nunc, 165306) except the background wells at four corners. After overnight incubation and the cells grew to the wall of the culture plate, the cells were intervened by 200 μmol/L hydrogen peroxide for 4 hours, then the medium containing hydrogen peroxide was removed, and a normal complete medium containing nucleotides with different concentrations (DMEM high-sugar medium containing 1% of penicillin/streptomycin and 10% of fetal bovine serum, and the concentrations of 5′-adenosine monophosphate and 5′-disodium uridine were respectively 100 μmol/L and 200 μmol/L, and the concentration of the nucleotide mixture was 200 μmol/L) was added for incubation for 24 hours. A probe panel (xFe96 FluxPak, Agilent, 102353-100) was hydrated with a hydration solution (XF Calibrant Solution, Agilent, 100840-000) one day in advance, and then was placed in a non-CO₂ incubator for incubation for at least 12 hours at 37° C. The experimental culture solution was prepared on the day of the experiment, 1 mL of 2.5 mM glucose (Glucose, Sigma, G7528), 1 mL of 2 mM glutamine (L-glutamine, Sigma, G8540), and 1 mL of 1 mM sodium pyruvate (Sodium pyruvate, Sigma, S8636) were added to 100 mL of base medium (Seahorse XF Basemedium, Agilent, 102353-100), and the pH of the culture solution was adjusted to 7.4±0.05 with 1N of NaOH. The culture solution of the cell culture plate was replaced with the experimental culture solution, 40 μL/well of the original medium was removed from the cell culture plate, then 160 μL/well of the experimental culture solution was added into the cell culture plate for dilution, and then 160 μL of the mixed culture solution was sucked out; the process was repeated for 2-3 times, finally the volume of the experimental culture solution in each well was 175 μL, and the cell culture plate was stood for 1 hour at 37° C. 25 μL of 2 μM oligomycin (Oligomycin, abcam, ab 141829) was added into well A of the probe panel, 25 μL of 1 μM carbonyl-cyano-p-trifluoromethoxyphenylhydrazine (FCCP, Sigma, C2920) was added into well B of the probe panel, and 25 μL of 1 μM Antimycin A (abcam, ab 141904)/Roteenone (Sigma, R8875) was added into well C of the probe panel. The probe panel was corrected on the machine, and the cell culture plate was detected on the machine. The blank control group was not intervened by 200 μmol/L of hydrogen peroxide and the model control group was intervened by 200 μmol/L of hydrogen peroxide, which were then added with normal complete medium.

The experimental methods in this example, unless otherwise specified, are conventional methods and are carried out in accordance with experimental methods or conditions described in the literature in the art or in accordance with the kit instructions. The materials, reagents, instruments and the like used in this example are commercially available. The experimental results are expressed by mean±standard deviation (x±SD). SPSS software is used to analyze the homoscedasticity of the data, and one-way analysis of variance (one-way ANOVA) is used for the homoscedasticity. The data with non-normal or uneven variance are performed with appropriate variable conversion, and statistics are performed after the data meet the requirements of normality or homoscedasticity. If the data still does not met the requirements after variable conversion, non-parametric test is used for statistics, and the least significant difference method (LSD) is used for comparison between the experimental group with the control group, with P<0.05 as the difference significance standard.

### II. Experimental Results of Examples 1-6

### 1. Effects of 5′-Monophosphate Nucleotides and Mixtures Thereof on Mitochondrial Oxygen Consumption Rate of Senescent Cells in Examples 1 and 2

The experimental results show that the oxygen consumption rate (OCR) of cells in the senescence group (model control group) decreased compared with the blank control group, the mitochondrial oxygen consumption of cells in the nucleotide mixture group (Example 2), AMP100 group and UMP200 group returned to normal level, and the basal respiration level of cells in the UMP200 group was significantly higher than that of the model control group (as shown in FIG. 1).

### 2. Effects of 5′-Monophosphate Nucleotides and Mixtures Thereof on Mitochondrial Basal Respiration of Senescent Cells in Examples 1 and 2

The experimental results show that the basal respiration level of cells in the senescence group (model control group) significantly decreased (P<0.05) compared with the blank control group, the basal level of cells in the nucleotide mixture group (Example 2), AMP100 group and UMP200 group returned to normal level, and the basal respiration level of cells in the UMP200 group was significantly higher than that of the model control group (P<0.05) (as shown in FIG. 2). In FIG. 2, # indicates that there was a significant difference compared with the blank control group, and * indicates that there was a significant difference compared with the model control group.

### 3. Effects of 5′-Monophosphate Nucleotides and Mixtures Thereof on Mitochondrial Maximum Respiratory Value of Senescent Cells in Examples 1 and 2

The experimental results show that the mitochondrial maximum respiratory value of H₂O₂-induced senescent cells in the model control group significantly decreased (P<0.05) compared with the blank control group; and compared with the model control group, the mitochondrial maximum respiratory value of cells in the nucleotide mixture group (Example 2), AMP100 group and UMP200 group significantly increased (P<0.05) (as shown in FIG. 3). In FIG. 3, #indicates that there was a significant difference compared with the blank control group, and * indicates that there was a significant difference compared with the model control group.

4. Effects of 5′-monophosphate nucleotides and mixtures thereof on mitochondrial ATP production capability of senescent cells in Examples 1 and 2

The experimental results show that the mitochondrial ATP production capability of cells in the model control group significantly decreased (P<0.05) compared with the blank control group; and compared with the model control group, the mitochondrial ATP production capability of cells in the nucleotide mixture group (Example 2), AMP100 group and UMP200 group significantly increased (P<0.05) (as shown in FIG. 4). In FIG. 4, #indicates that there was a significant difference compared with the blank control group, and * indicates that there was a significant difference compared with the model control group.

### 5. Effects of 5′-Monophosphate Nucleotides and Mixtures Thereof on Mitochondrial Respiratory Reserve of Senescent Cells in Examples 1 and 2

The experimental results show that the mitochondrial respiratory reserve value of cells in the model control group significantly decreased (P<0.05) compared with the blank control group; and compared with the model control group, the mitochondrial respiratory reserve value of cells in the nucleotide mixture group (Example 2), AMP100 group and UMP200 group significantly increased (P<0.05) (as shown in FIG. 5). In FIG. 5, #indicates that there was a significant difference compared with the blank control group, and * indicates that there was a significant difference compared with the model control group.

### 6. Effects of 5′-Monophosphate Nucleotides and Mixtures Thereof on Mitochondrial Maximum Respiratory Value of Senescent Cells in Examples 3 to 6

The experimental results show that the mitochondrial maximum respiratory value of H₂O₂-induced senescent cells in the model control group significantly decreased (P<0.05) compared with the blank control group; and compared with the model control group, the mitochondrial maximum respiratory value of cells in the nucleotide mixture group in Examples 3 to 6 significantly increased (P<0.05) (as shown in FIG. 6). In FIG. 6, # indicates that there was a significant difference compared with the blank control group, and * indicates that there was a significant difference compared with the model control group.

### III. Experimental Conclusions

Taking the blank control group and model control group as control groups, the present invention explores the effects of two 5′-monophosphate nucleotides and the mixtures thereof on improving mitochondrial function. Cell experiments prove that the said two 5′-monophosphate nucleotides and the mixtures thereof can significantly improve the basal respiration, maximum respiratory value, ATP production capability and respiratory reserve of mitochondria of the H₂O₂-induced senescent cells, indicating that the said two 5′-monophosphate nucleotides and the mixtures thereof have significant effect on improving mitochondrial function and have the potential to be used as a novel drug and food for improving mitochondrial function.

### Example 8

### I. Materials and Methods

### 1. Samples

The nucleotide mixture samples obtained in the Example 2.

### 2. Groups and Dosages

In this example, the concentration of the nucleotide mixture obtained in Example 2 was 200 μmol/L, and was recorded as the nucleotide mixture group. Model control group was a senescence cell model group intervened by hydrogen peroxide. Blank control group was a cell model group free of 5′-adenosine monophosphate, 5′-disodium uridine and a nucleotide mixture without hydrogen peroxide intervention. The single nucleotide species was 5′-cytidine monophosphate, 5′-disodium guanylate, and 5′-disodium inosinate, with a concentration of 200 μmol/L, respectively.

### 3. Experimental Cells

The cells used in the example were mouse embryonic fibroblasts NIH/3T3, which were purchased from Cell Bank of Chinese Academy of Sciences.

### 4. Establishment of Senescence Cell Model

The cells were cultured in a DMEM high-sugar medium containing 1% penicillin/streptomycin and 10% fetal bovine serum in an incubator with 5% of CO₂ at 37°° C. under saturated humidity conditions. The establishment of the senescence cell model was as follows: After the cells grew to the wall of a culture vessel, different concentrations of hydrogen peroxide were used for intervention, where the concentrations of hydrogen peroxide were 50 μmol/L, 100 μmol/L, 200 μmol/L, 400 μmol/L, and 800 μmol/L. The medium containing hydrogen peroxide was removed after incubation for 4 hours, and a normal complete medium (DMEM high-sugar medium containing 1% penicillin/streptomycin and 10% fetal bovine serum) was added for incubation for 24 hours, then the cell activity of the cultured cells was detected by using a CCK-8 cell active kit, the expression of β-galactosidase was detected, and the expressions of the senescent marker p^{16INK4A} and p^{21Waf1/Cip1} were detected by western blot method. According to the senescent result, 200 μmol/L was selected as the optimal concentration of hydrogen peroxide for subsequent experiments.

### 5. Experimental Method

In this example, the oxygen consumption rate (OCR), basal respiration, maximum respiratory value, ATP production capability and respiratory reserve of mitochondria were determined by using the Seahorse xFe96 (Agilent) mitochondria stress detection experiment. 20000 NIH/3T3 cells were inoculated in each well of a 96-well cell culture plate (Nunc, 165306) except the background wells at four corners. After overnight incubation and the cells grew to the wall of the culture plate, the cells were intervened by 200 μmol/L hydrogen peroxide for 4 hours, then the medium containing hydrogen peroxide was removed, and a normal complete medium containing nucleotides with different concentrations (DMEM high-sugar medium containing 1% of penicillin/streptomycin and 10% of fetal bovine serum, and the concentration of the nucleotide mixture was 200 μmol/L, and the concentration of each of 5′-cytidine monophosphate, 5′-disodium guanylate, and 5′-disodium inosinate was 200 μmol/L,) was added for incubation for 24 hours. A probe panel (xFe96 FluxPak, Agilent, 102353-100) was hydrated with a hydration solution (XF Calibrant Solution, Agilent, 100840-000) one day in advance, and then was placed in a non-CO₂ incubator for incubation for at least 12 hours at 37° C. The experimental culture solution was prepared on the day of the experiment, 1 mL of 2.5 mM glucose (Glucose, Sigma, G7528), 1 mL of 2 mM glutamine (L-glutamine, Sigma, G8540), and 1 mL of 1 mM sodium pyruvate (Sodium pyruvate, Sigma, S8636) were added to 100 mL of base medium (Seahorse XF Basemedium, Agilent, 102353-100), and the pH of the culture solution was adjusted to 7.4±0.05 with 1N of NaOH. The culture solution of the cell culture plate was replaced with the experimental culture solution, 40 μL/well of the original medium was removed from the cell culture plate, then 160 μL/well of the experimental culture solution was added into the cell culture plate for dilution, and then 160 μL of the mixed culture solution was sucked out; the process was repeated for 2-3 times, finally the volume of the experimental culture solution in each well was 175 μL, and the cell culture plate was stood for 1 hour at 37° C. 25 μL of 2 μM oligomycin (Oligomycin, abcam, ab 141829) was added into well A of the probe panel, 25 μL of 1 μM carbonyl-cyano-p-trifluoromethoxyphenylhydrazine (FCCP, Sigma, C2920) was added into well B of the probe panel, and 25 μL of 1 μM Antimycin A (abcam, ab 141904)/Roteenone (Sigma, R8875) was added into well C of the probe panel. The probe panel was corrected on the machine, and the cell culture plate was detected on the machine. The blank control group was not intervened by 200 μmol/L of hydrogen peroxide and the model control group was intervened by 200 μmol/L of hydrogen peroxide, each of which was then added with normal complete medium, respectively.

The experimental methods in this example, unless otherwise specified, are conventional methods and are carried out in accordance with experimental methods or conditions described in the literature in the art or in accordance with the kit instructions. The materials, reagents, instruments and the like used in this example are commercially available. The experimental results are expressed by mean±standard deviation (x±SD). SPSS software is used to analyze the homoscedasticity of the data, and one-way analysis of variance (one-way ANOVA) is used for the homoscedasticity. The data with non-normal or uneven variance are performed with appropriate variable conversion, and statistics are performed after the data meet the requirements of normality or homoscedasticity. If the data still does not met the requirements after variable conversion, non-parametric test is used for statistics, and the least significant difference method (LSD) is used for comparison between the experimental group with the control group, with P<0.05 as the difference significance standard.

II. Experimental results

### 1. Effects of Nucleotide Mixture and Single Nucleotide Species (CMP, GMP, IMP) on Mitochondrial Basal Respiration of Senescent Cells in Example 2

The experimental results show that the basal respiration level of cells in the model control group significantly decreased (P<0.05) compared with the blank control group; and compared with the module control group, the basal level of cells in the nucleotide mixture group (Example 2) returned to normal level, and the basal respiration level of cells in the CMP, GMP and IMP groups increased and significantly lower than that in the nucleotide mixture group (P<0.05) (as shown in FIG. 7). In FIG. 7, #indicates that there was a significant difference compared with the blank control group, and * indicates that there was a significant difference compared with the model control group.

### 2. Effects of Nucleotide Mixture and Single Nucleotide Species (CMP, GMP, IMP) on Mitochondrial Maximum Respiratory Value of Senescent Cells in Example 2

The experimental results show that the mitochondrial maximum respiratory value of H₂O₂-induced senescent cells in the model control group significantly decreased (P<0.05) compared with the blank control group; and compared with the model control group, the mitochondrial maximum respiratory value of cells in each the CMP, GMP and IMP groups increased (P<0.05) but significantly lower than that in the nucleotide mixture group (Example 2) (P<0.05) (as shown in FIG. 8). In FIG. 8, #indicates that there was a significant difference compared with the blank control group, and * indicates that there was a significant difference compared with the model control group.

### 3. Effects of Nucleotide Mixture and Single Nucleotide Species (CMP, GMP, IMP) on Mitochondrial ATP Production Capability of Senescent Cells in Example 2

The experimental results show that the mitochondrial ATP production capability of cells in the model control group significantly decreased (P<0.05) compared with the blank control group; and compared with the model control group, the mitochondrial ATP production capability of cells in each the CMP, GMP and IMP groups increased (P<0.05) but significantly lower than that in the nucleotide mixture group (Example 2) (P<0.05) (as shown in FIG. 9). In FIG. 9, #indicates that there was a significant difference compared with the blank control group, and * indicates that there was a significant difference compared with the model control group.

### 4. Effects of Nucleotide Mixture and Single Nucleotide Species (CMP, GMP, IMP) on Mitochondrial Respiratory Reserve of Senescent Cells in Example 2

The experimental results show that the mitochondrial respiratory reserve value of cells in the model control group significantly decreased (P<0.05) compared with the blank control group; and compared with the model control group, the mitochondrial respiratory reserve value of cells in each the CMP, GMP and IMP groups increased (P<0.05) but significantly lower than that in the nucleotide mixture group (Example 2) (P<0.05) (as shown in FIG. 10). In FIG. 10, #indicates that there was a significant difference compared with the blank control group, and * indicates that there was a significant difference compared with the model control group.

### III. Experimental Conclusions

Taking the blank control group and model control group as control groups, the present invention explores the effects of the nucleotide mixture and single nucleotide species (CMP, GMP, IMP) on improving mitochondrial function. Cell experiments prove that the nucleotide mixture can significantly improve the basal respiration, maximum respiratory value, ATP production capability and respiratory reserve of mitochondria of the H₂O₂-induced senescent cells, and single CMP, GMP and IMP had little effect on the basal respiration, maximum respiratory value, ATP production capability and respiratory reserve of mitochondria of the H₂O₂induced senescent cells, indicating that 5′-monophosphate nucleotide mixture has significant effect on improving mitochondrial function.

For those skilled in the art, without departing from the scope of the technical solution of the present invention, many possible changes and modifications can be made to the technical solution of the present invention by using the technical contents disclosed above, or modified into equivalent embodiments with equivalent changes. Therefore, any simple modification, equivalent change and modification made to the above embodiments according to the technical essence of the present disclosure without departing from the technical solution of the present invention shall still belong to the protection scope of the technical solution of the present invention.

1. A drug or food item for improving mitochondrial function comprising 5′-monophosphate nucleotide, wherein the 5′-monophosphate nucleotide is 5′-adenosine monophosphate or 5′-disodium uridine.

2. A drug or food item for improving mitochondrial function, comprising a 5′-monophosphate nucleotide, wherein the nucleotide mixture comprises four or five 5′ mononucleotides or sodium salts thereof, and mass percentages of the various nucleotides calculated according to forms of CMP, AMP, UPMP, GMP, IMP are respectively as follows: 23-78% of CMP, 6-44% of AMP, 7 -40% of UMP, 7-51% of GMP, and IMP being 0 or greater than 0 and not greater than 2.5%.

3. The drug or food item according to claim 1, wherein each of the 5′-adenosine monophosphate and 5′-disodium uridine is produced by means of enzymatic degradation of a ribonucleic acid as a raw material and has a purity of more than 99%.

4. The drug or food item according to claim 2, wherein mass percentages of the various nucleotides calculated according to forms of CMP, AMP, UPMP, GMP, IMP are respectively as follows: 25.80% of CMP, 22.80% of AMP, 20.40% of UMP, 30.20% of GMP and 0.8% of IMP.

5. The drug according to claim 1, wherein the drug is in the forms of powders, tablets, soft capsules, hard capsules, or oral liquids.

6. The food item according to claim 1, wherein the food item is in the forms of powders or liquid beverages, preferably in the forms of milk powders, dairy products, or bakery products.

7. The drug or food item according to claim 1, wherein the 5′-monophosphate nucleotide and mixture thereof improve a basal respiration, a maximum respiratory value, an ATP production capability, and a respiratory reserve of the mitochondria of H2O2-induced senescent cells.

8. The drug according to claim 2, wherein the drug is in the forms of powders, tablets, soft capsules, hard capsules, or oral liquids.

9. The food item according to claim 2, wherein the food item is in the forms of powders or liquid beverages, preferably in the forms of milk powders, dairy products, or bakery products.
